(19) United States
(12) Patent Application Publication
(10) Pub. No.: US 2023/0301571 A1
(43) Pub. Date: Sep. 28, 2023

(54) AI TEST AND ANALYSIS SYSTEM FOR PSYCHOLOGY PREFERENCE

(71) Applicants: Ching Lee, Taipei City (TW); Ruey Yuan Lee, Taipei City (TW)

(72) Inventors: Ching Lee, Taipei City (TW); Ruey Yuan Lee, Taipei City (TW)

(21) Appl. No.: 17/702,821

(22) Filed: Mar. 24, 2022

(51) Int. Cl. A61B 5/16; A61B 5/369; A61B 5/00

(52) U.S. Cl. CPC A61B 5/165; A61B 5/369; A61B 5/162; A61B 5/7264; A61B 5/6803; A61B 5/0006

(57) ABSTRACT

An AI test and analysis system for psychology preference is provided. By using brainwave detectors, the emotions and characters of the testers are connected. Variations from the brainwaves in pre-test and formal-test are used for analyzing of AI and big data as a base for determining human characters and emotions. These can be used to compensate insufficiency of conventional analysis thereabout. The methodology is widely used in various fields, such as mindfulness, human resource, potentials of people, and sensibilities of human, etc. Only data obtained from brainwave detectors are used to have tendencies and preference of the testers to various objects, while brainwaves are real physical data from the testers.

### FIELD OF THE INVENTION

The present invention is related to brainwave application to human characters, and in particular to an AI test and analysis system for psychology preference.

### BACKGROUND OF THE INVENTION

Currently, from the research of brainwaves, it is knows that the tightness, hearing ability, memory, logical ability, visual ability, reaction can be acquired from analysis of brainwaves. The brainwaves of human mainly include Delta wave, Theta waves, High/Low Alpha waves, High/Low Beta waves and High/Low Gamma waves of the left and right brains of the testers. These brainwaves have different physical, physiological, and psychological meanings, which expresses different conditions of the testers. Therefore, by measuring brainwaves and numerical operations thereto, characters and emotions of the testers can be got. These have been widely and deeply researched academically. The operations are executed in relative semiconductor chips.

That Delta wave, Theta waves, High/Low Alpha waves, High/Low Beta waves and High/Low Gamma waves of the left and right brains represents the characters of the testers have been widely accepted medically. Research and creation are related to the strengths of Delta waves and Low Alpha waves; wisdom, logics, calming down are related to the strength of Delta waves, High/Low Alpha waves; carefulness and concentration are related to the strengths of High Alpha waves and Low Beta waves; liveliness, affinity are related to the strengths of High/Low Beta waves; reaction and willfulness are related to the strengths of High/Low Gamma waves. Normally, variations of brainwaves measured continuously are randomly.

Recently, concept of Metaverse is abrupt popular all over the world. One of the important features in Metaverse is how to imitate the human characters so as to make the virtual world more approaches the real world. It is known that Helmet brainwaves detectors can get the states of the brainwaves real time. By serial tests about emotions, reactions, and preference to find characters of human is a big trend in research of brainwaves. For example, Theta waves and Low Alpha waves are helpful to find the creations and inspiration of humans.

Therefore, since inventors of the present invention have worked in this field for many years and owns plentiful professional knowledge about these fields, they desire to propose a novel method which combines characters of brainwaves and digital coding systems so as to resolve problems encountered in the prior art.

### SUMMARY OF THE INVENTION

Accordingly, the object of the present invention is to provide an AI test and analysis system for psychology preference, wherein by using brainwave detectors, the emotions and characters of the testers are connected. Variations from the brainwaves in pre-test and formal-test are used for analyzing of AI and big data as a base for determining human characters and emotions. These can be used to compensate insufficiency of conventional analysis thereabout. The methodology of the present invention can be widely used in various fields, such as mindfulness, human resource, potentials of people, and sensibilities of human, etc. Only data obtained from brainwave detectors are used to have tendencies and preference of the testers to various objects, while brainwaves are real physical data from the testers, therefore, what get from the present invention are very near the realities of the testers.

To achieve above object, the present invention provides an AI test and analysis system for psychology preference including: a helmet for detection of the brainwave of a tester; the helmet including a heat ring, a brainwave detector in the head ring and a brainwave transceiver connected to the brainwave detector for transmitting brainwaves from the brainwave detector outwards; a processing unit connected to the helmet for receiving the brainwaves for performing AI processing and comparing brainwaves for acquiring associated information. The processing unit comprises a processing end transceiver signally connected the brainwave receiver for receiving brainwaves therefrom; a test and analyzing unit connected to the processing end transceiver, which receives brainwaves of the testers and then analyzes the brainwaves by using big data technologies; the features of the brainwaves being determined by predetermined algorithm for determining reaction of preference of the testers for specific objects; a plurality of testers being tested for the same test so as to find common brainwave features and then build a brainwave feature standard module; a brainwave contrast module connected to the processing end transceiver and the test and analyzing unit for contrasting the brainwaves from another tester which is contrasted with the brainwave feature standard module so as to determine the preference of the tester to a testing object, as a result, various tendencies of the tester can be known.

### BRIEF DESCRIPTION OF THE DRAWINGS

FIG. 1 shows a block diagram showing the main components of the present invention.

FIG. 2 shows the helmet of the present invention.

FIG. 3 is a schematic view showing the test flow diagram and the application of the present invention.

FIG. 4 is a schematic view showing the test process of the present invention.

FIG. 5 shows that the present invention is applied to mindfulness estimation.

FIG. 6 shows that the present invention is utilized to human resource estimation.

FIG. 7 is a schematic view showing that the present invention is applied to potential estimation.

FIG. 8 is a schematic view showing that the present invention is utilized to Human sensibility and estimation of thought.

### DETAILED DESCRIPTION OF THE INVENTION

In order that those skilled in the art can further understand the present invention, a description will be provided in the following in details. However, these descriptions and the appended drawings are only used to cause those skilled in the art to understand the objects, features, and characteristics of the present invention, but not to be used to confine the scope and spirit of the present invention defined in the appended claims.

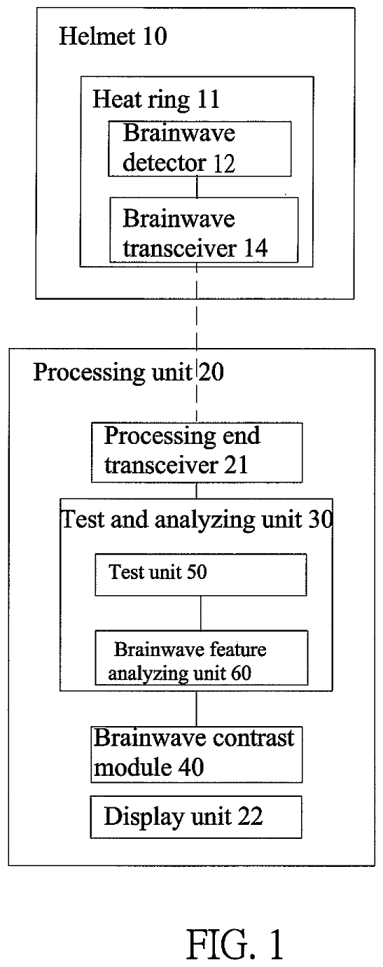

Referring to FIGS. 1 to 4, the structure of the present invention is illustrated. The present invention includes the following elements.

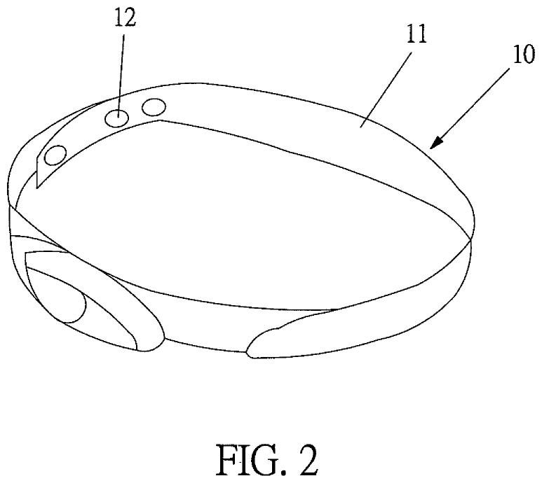

A helmet 10 is utilized for detection of the brainwave of a tester. In utilization, the helmet 10 is worn on the head of the tester, as illustrated in FIGS. 1 and 2. The helmet includes a heat ring 11, a brainwave detector 12 in the head ring 11 and a brainwave transceiver 14 connected to the brainwave detector 12 for transmitting brainwaves from the brainwave detector 12 outwards.

A processing unit 20 is connected to the helmet 10 for receiving the brainwaves for performing AI (artificial intelligent) processing and comparing brainwaves before test and after test for acquiring associated information.

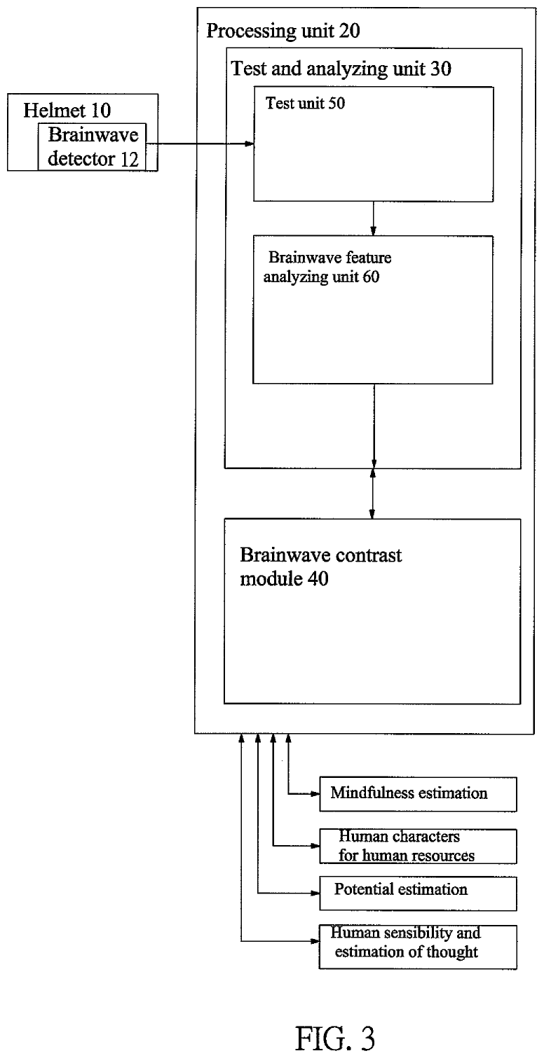

The processing unit 20 receives a series of brainwaves from the helmet 10, including brainwaves from the left brain and right brain of the tester, which contains Delta wave, Theta waves, High/Low Alpha waves, High/Low Beta waves and High/Low Gamma waves, as shown in FIGS. 1 and 3.

The processing unit 20 comprises:

A processing end transceiver 21 signally connected the brainwave receiver 14 for receiving brainwaves therefrom.

A test and analyzing unit 30 connected to the processing end transceiver 21, which receives brainwaves of the testers and then analyzes the brainwaves by using big data technologies. The features of the brainwaves are determined by predetermined algorithm for determining reaction of preference of the testers for specific objects. A plurality of testers are tested for the same test so as to find common brainwave features and then build a brainwave feature standard module 31. This is mainly from the variation of the Delta wave, Theta waves, High/Low Alpha waves, High/Low Beta waves and High/Low Gamma waves of the left and right brains of the testers.

A brainwave contrast module 40 is connected to the processing end transceiver 21 and the test and analyzing unit 30, and contrast the brainwaves from another tester which is contrasted with the brainwave feature standard module 31 so as to determine the preference of the tester to a testing object, as a result, various tendencies of the tester can be known.

A display unit 22 connected to the processing unit 20 serves to display the test result.

The processing unit 20 is able to be installed to various electronic devices, such as computers, mobile phones, tablet computers, etc.

The test and analyzing unit 30 comprises:

A test unit 50 performs tests to testers. The test may include pre-tests and formal-test. The pre-test means to receive the brainwaves of the tester before the tester does not give any object for testing; and the formal-test means to provide an object to the tester, then receiving the responding brainwaves from the testers

A brainwave feature analyzing unit 60 receives brainwave signals from the testers and response from the testers, and analyzes the features of the brainwave signals.

The test unit 50 receives responses of the testers to the testing objects. The responses are the preferences of the objects (means that the testers like or unlike the testing objects), it being classified into five grades, including very like, like, no feeling, dislike, and very dislike. Then the brainwave feature analyzing unit 60 analyzes features of the brainwaves of the testers due to the response from the tester to the testing objects. The method is to capture the brainwaves of the testers in various time stages when the tester is tested by sensing the testing objects; then to find variations of the received brainwaves from the testers, some sections of the brainwaves existing continuous variations (increase or decrease along the same direction) being considered to be the features about the preference of the tester. The time periods of the changes of strengths in the brainwaves are detected. This work is executed for various brainwaves and different sections of the brainwaves for various different persons for acquiring features of brainwaves.

Generally, variations of brainwaves captured in continuous different time periods are randomly distributed. However, if the variations for different time periods have identical trends, these variations are deemed as a feature of the brainwaves. The greater the change of the strength of the brainwaves, the obvious the variation of the feature. By combining the trend and strength, the psychological state of the tester is judged.

The above mentioned captured brainwaves includes brainwaves from pre-test and formal test, and brainwave under mindfulness. The test and analyzing unit 30 determines these brainwaves to acquire features.

The test and analyzing unit 30 classifies the eight sections of brainwaves into three classes. A first class includes Delta waves, and Theta waves which expresses natures and talents; a second classes includes Theta waves, and High/Low Alpha waves which express consciousness of human brain; and a third class includes High/Low Alpha waves, High/Low Beta waves and High/Low Gamma waves which expresses personalities. These classes are tested and analyzed.

Abilities about Research and creation are related to the strengths of Delta waves and Low Alpha waves; wisdom, logics, calming down of the emotion are related to the strengths of Delta waves, and High/Low Alpha waves; carefulness and concentration are related to the strengths of High Alpha waves and Low Beta waves; liveliness, and affinity are related to the strengths of High/Low Beta waves; and reaction and willfulness are related to the strengths of High/Low Gamma waves.

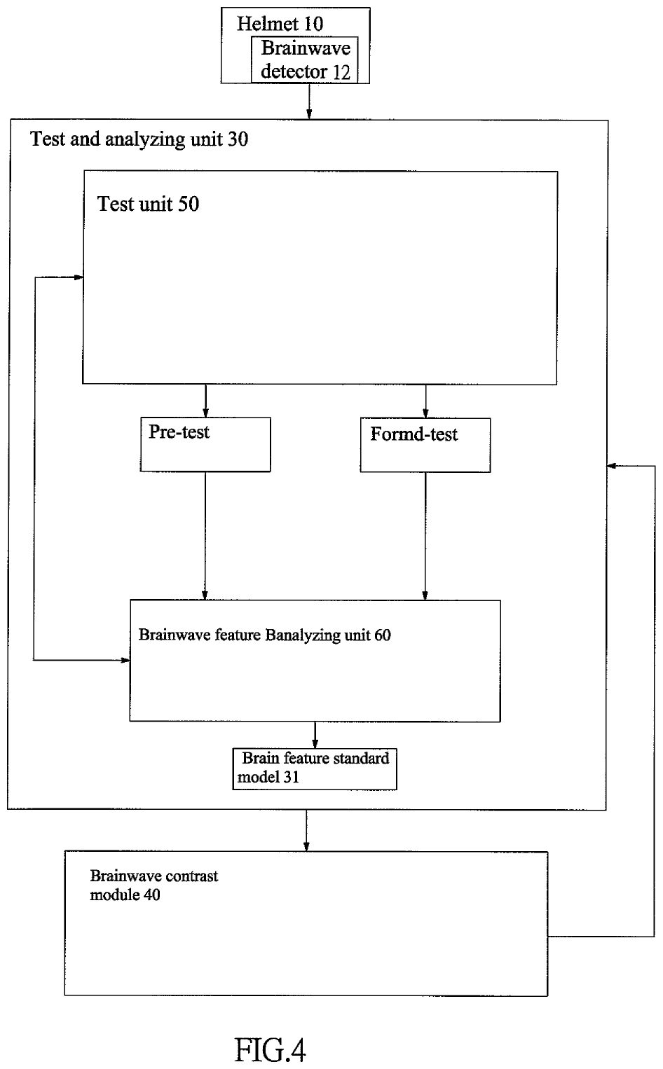

Referring to FIG. 4, the test and analyzing unit 30 tests the tester about the following items, that includes

Olfaction test using a plurality of tasteful objects (such as essences) as test objects;

Visual test using a plurality of plots and videos as test objects;

Taste sense test using a plurality of foods as objects;

Hearing teat using a plurality of voices (such as music) as test object;

Touch sense test using a plurality of objects as test objects;

Class test using a plurality of professional classes as test objects; and

Communication test being executed by a test set of two personas. They perform the tests of viewing each other silently, talking to each other and talking with handcrafts.

Above tests are executed to many people so as to obtain a brain feature standard model 31 which is suitable to all people and represents brain features of preference of a person to a specific object. The brain feature standard model 31 is used to acquire the preference of the other tester to some specific testing object by comparing the brainwaves of the other tester with those recording in the brain feature standard model 31. Therefore, by controlling the testing objects, various tendencies of the other tester can be known.

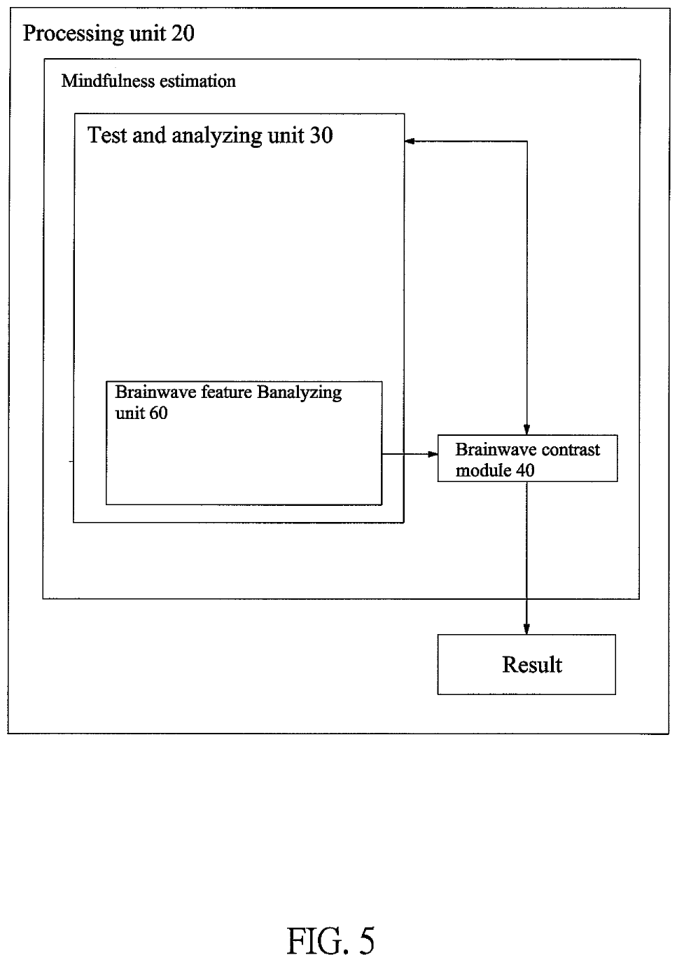

With reference to FIGS. 5 to 8, the processing unit 20 of the present invention can be used to various personality traits estimations, which includes the following items.

1. Mindfulness estimation: as illustrated in FIG. 5, no pre-test is performed, just the formal test is performed, in that the tester is in a state of mindfulness (in a state that no any ideal appears in the mind). At this time period, the brainwaves of the tester are in lowest amplitudes with lowest power consumption. The brainwaves in mindfulness are compared with those in normal state (before testing) by the average power valve of different sections of the brainwaves. By this way, potentials of the tester can be acquired. Furthermore big data analysis is applied to calculate concentration, tightness, and strengths of various sections of the brainwaves to acquire reports about the mindfulness.

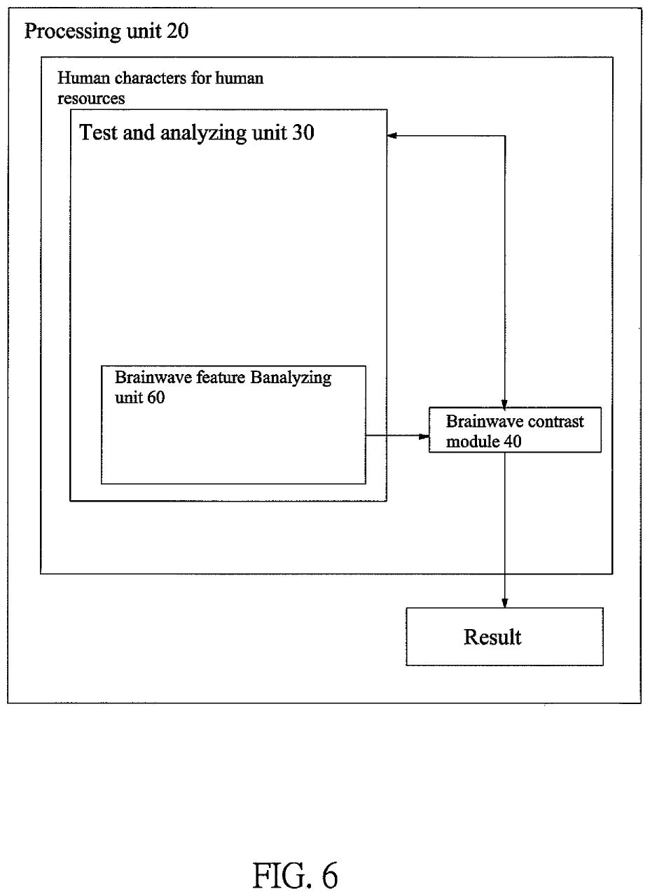

2. Human characters for human resources, as illustrated in FIG. 6, can be obtained. In pre-test, data about specific classes are read and in formal-test, mindfulness is tested. The brainwaves for pre-test and formal-test are executed for big data analysis. Various sections of the brainwaves are analyzed for acquiring human characters of the testers which are used in human resources (such as leading ability, abilities of creation and development, integration and research, administration, business, and marketing, production and managements, etc.).

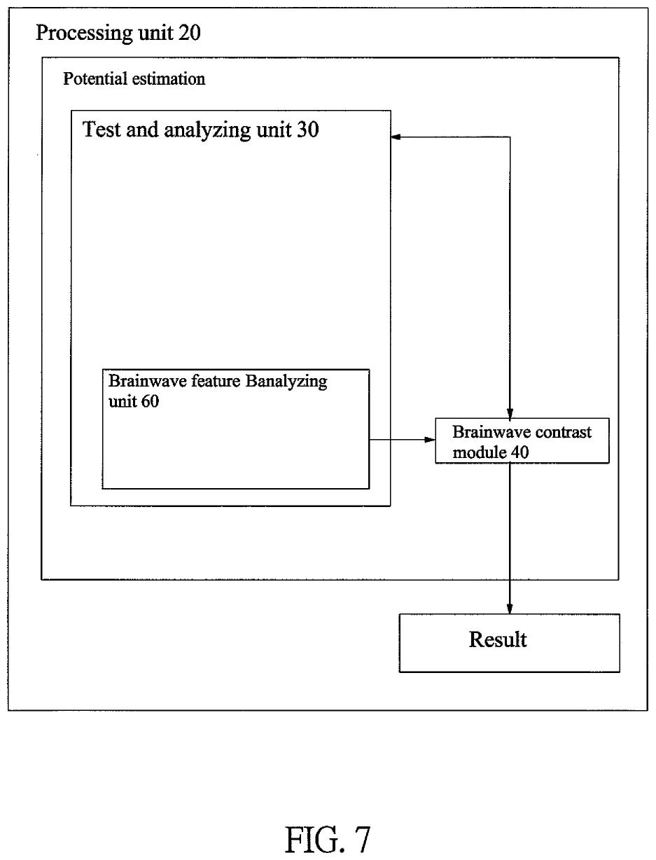

3. Potential estimation, as illustrated in FIG. 7, is acquired by the present invention. In pre-test, data about specific classes are read and in formal-test, mindfulness is tested. The brainwaves for pre-test and formal-test are executed for big data analysis. Various sections of the brainwaves are analyzed for acquiring human characters of the testers which are used in acquiring potentials of the tester (such as potentials in natural science, arts, logic judge, memory, calculation, social ability, endurance, wisdom and reactions, etc.).

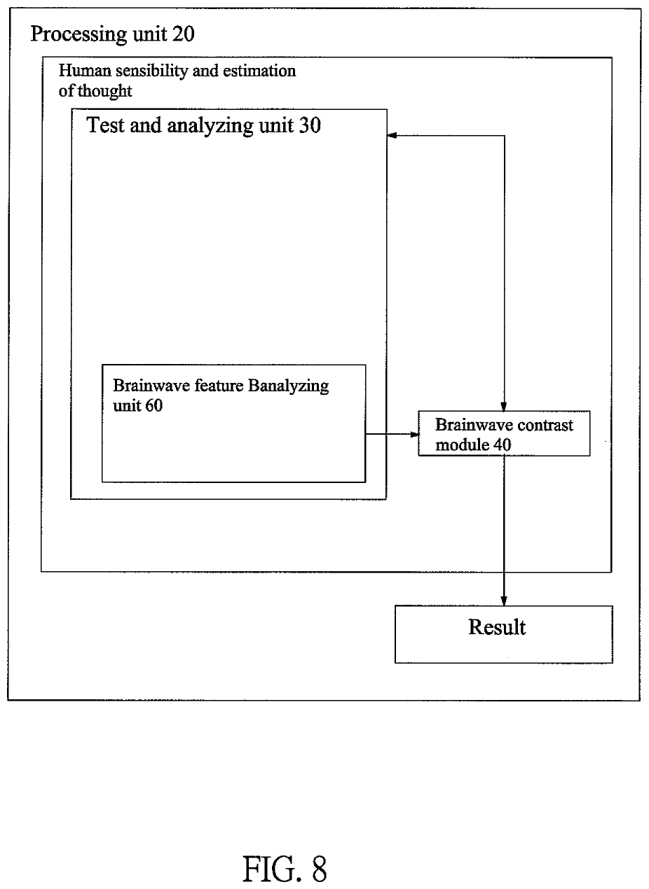

4. Human sensibility and estimation of thought, as illustrated in FIG. 8, are acquired. In that, no pre-test is performed, In formal-test, tests about olfaction, visual, taste, hearing, touch, and thought of brain are executed, by such as sensing tasteful objects (such as essences), plots and videos, foods, music, touching of objects, learning of professional classes; and communication with others. Big data analysis is performed for acquiring a module to determine emotion and logics of a tester.

Advantages of the present invention are that: by using brainwave detectors, the emotions and characters of the testers are connected. Variations from the brainwaves in pre-test and formal-test are used for analyzing of AI and big data as a base for determining human characters and emotions. These can be used to compensate insufficiency of conventional analysis thereabout. The methodology of the present invention can be widely used in various fields, such as mindfulness, human resource, potentials of people, and sensibilities of human, etc. Only data obtained from brainwave detectors are used to have tendencies and preference of the testers to various objects, while brainwaves are real physical data from the testers, therefore, what get from the present invention are very near the realities of the testers.

The present invention is thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. An AI test and analysis system for psychology preference including:
a helmet for detection of brainwaves of testers; the helmet including a brainwave detector and a brainwave transceiver connected to the brainwave detector for transmitting brainwaves from the brainwave detector;
a processing unit connected to the helmet for receiving the brainwaves for performing AI processing and comparing brainwaves for acquiring associated information;
the processing unit comprising:
a processing end transceiver signally connected the brainwave receiver for receiving brainwaves therefrom;
a test and analyzing unit connected to the processing end transceiver, which receives brainwaves of the testers and then analyzes the brainwaves by using big data technologies; the features of the brainwaves being determined by predetermined algorithm for determining reaction of preference of the testers for specific objects; a plurality of testers being tested for the same test so as to find common brainwave features and then build a brainwave feature standard module; and
a brainwave contrast module connected to the processing end transceiver and the test and analyzing unit for contrasting the brainwaves from another tester which is contrasted with the brainwave feature standard module so as to determine the preference of the tester to a testing object, as a result, various tendencies of the tester can be known.

2. The AI test and analysis system for psychology preference as claimed in claim 1, wherein the test and analyzing unit classifies the eight sections of brainwaves into three classes; and the test and analysis is based on these three classes.

3. The AI test and analysis system for psychology preference as claimed in claim 1, wherein the test and analyzing unit comprises;
a test unit performing tests to testers; the test includes pre-tests and formal-test; the pre-test means to receive the brainwaves of the tester before the tester does not give any object for testing; and the formal-test means to provide an object to the tester, then receiving the responding brainwaves from the testers; and
a brainwave feature analyzing unit receiving brainwave signals from the testers and response from the testers, and analyzes the features of the brainwave signals.

4. The AI test and analysis system for psychology preference as claimed in claim 1, wherein the test unit receives responses of the testers to the testing objects; the responses are the preferences of the objects, that is: the testers like or unlike the testing objects; it being classified into several grades; then the brainwave feature analyzing unit analyzes features of the brainwaves of the testers due to the response from the tester to the testing objects;
wherein the method is to capture the brainwaves of the testers in various time stages when the tester is tested by sensing the testing objects; then to find variations of the received brainwaves from the testers, some sections of the brainwaves existing continuous variations, that is: increase or decrease along the same direction, being considered to be the features about the preference of the tester.

5. The AI test and analysis system for psychology preference as claimed in claim 4, wherein the above mentioned captured brainwaves includes brainwaves from pre-test and formal test, and brainwave under mindfulness; and the test and analyzing unit determines these brainwaves to acquire features.

6. The AI test and analysis system for psychology preference as claimed in claim 5, wherein the test and analyzing unit tests the tester about the following items, that includes:
Olfaction test using a plurality of tasteful objects as test objects;
Visual test using a plurality of plots and videos as test objects;
Taste sense test using a plurality of foods as objects;
Hearing teat using a plurality of voices as test object;
Touch sense test using a plurality of objects as test objects;
Class test using a plurality of professional classes as test objects; and
Communication test being executed by a test set of two personas; they perform the tests of viewing each other silently, talking to each other and talking with handcrafts.

7. The AI test and analysis system for psychology preference as claimed in claim 1, wherein a Mindfulness estimation is performed, in that: no pre-test is performed, just the formal test is performed, wherein the tester is in a state of mindfulness, i. e., in a state that no any ideal appears in the mind; the brainwaves in mindfulness are compared with those in normal state before testing; by this way, potentials of the tester can be acquired; furthermore big data analysis is applied to calculate concentration, tightness, and strengths of various sections of the brainwaves to acquire reports about the mindfulness.

8. The AI test and analysis system for psychology preference as claimed in claim 1, wherein a Human characters for human resources is executed, in pre-test, data about specific classes are read and in formal-test, mindfulness is tested; the brainwaves for pre-test and formal-test are executed for big data analysis; various sections of the brainwaves are analyzed for acquiring human characters of the testers which are used in human resources.

9. The AI test and analysis system for psychology preference as claimed in claim 1, wherein a potential estimation is performed, in pre-test, data about specific classes are read and in formal-test, mindfulness is tested; the brainwaves for pre-test and formal-test are executed for big data analysis; various sections of the brainwaves are analyzed for acquiring human characters of the testers which are used in acquiring potentials of the tester.

10. The AI test and analysis system for psychology preference as claimed in claim 1, wherein a Human sensibility and estimation of thought are acquired; in that, no pre-test is performed, in formal-test, tests about olfaction, visual, taste, hearing, touch, and thought of brain are executed, by such as sensing tasteful objects, plots and videos, foods, music, touching of objects, learning of professional classes; and communication with others; and big data analysis is performed for acquiring a module to determine emotion and logics of a tester.
